(19) United States
(12) Patent Application Publication
(10) Pub. No.: US 2023/0157254 A1
(43) Pub. Date: May 25, 2023

(54) METHODS OF MAKING FUNGUS ORGANISM HYBRIDS WITH NOVEL OR ENHANCED PHYTOCHEMICALS

(71) Applicant: Blue Sun Mycology Group, LLC, Lebanon, OR (US)

(72) Inventor: Jerred Tudela, Lebanon, OR (US)

(21) Appl. No.: 17/990,573

(22) Filed: Nov. 18, 2022

Related U.S. Application Data
(60) Provisional application No. 63/281,532, filed on Nov. 19, 2021.

(51) Int. Cl. A01H 15/00; A01G 18/00

(52) U.S. Cl. CPC A01H 15/00; A01G 18/00

(57) ABSTRACT

Methods of crossbreeding dikaryotic fungus organisms to yield a hybrid fungus organism with a novel or enhanced phytochemical. The methods include providing a first growth medium; placing a first dikaryotic fungus organism with a first phytochemical on the first growth medium; placing a second dikaryotic fungus organism with a second phytochemical on the first growth medium adjacent to the first dikaryotic fungus organism; allowing the first dikaryotic fungus organism to replicate and to form a first colony on the first growth medium; allowing the second dikaryotic fungus organism to replicate and to form a second colony on the first growth medium; and allowing the first colony and the second colony to expand until they intersect along a clamp line where the first colony and the second colony exchange genetic material between them to yield a hybrid fungus organism with a novel or enhanced phytochemical.

### CROSS REFERENCE TO RELATED APPLICATIONS

This application claims priority to copending U.S. Application. Ser. No. 63/281,532, filed on Nov. 19, 2021, which is hereby incorporated by reference for all purposes.

### BACKGROUND

The present disclosure relates generally to fungus organisms and methods of crossbreeding fungus organisms. In particular, methods of making fungus organism hybrids with novel or enhanced phytochemicals are described.

Phytochemicals are secondary metabolite compounds produced by plant and fungus species in nature. Phytochemicals can be extracted from plant and fungus species and used for industrial, health, medicinal, or recreational purposes. Some of the phytochemicals found in plants and fungi are alkaloids, flavonoids, glycosides, and phenols. It is believed that these phytochemicals help the body ward off toxins, recover from illness, and alleviate pain.

While naturally occurring phytochemicals have many beneficial properties, it would be desirable to produce fungus organisms with novel or enhanced phytochemicals to provide even more industrial, health, medicinal, or recreational benefits. Novel phytochemicals are phytochemicals that do not exist in nature. Enhanced phytochemicals are phytochemicals already known to exist, but at higher potency or in a higher concentration than they exist in nature. There would be advantages to fungus organisms with novel or enhanced phytochemicals tailored to provide certain benefits, such as increased treatment potential for a given disease or condition.

Thus, there exists a need for fungus organisms with novel or enhanced phytochemicals to improve on conventional ways of acquiring phytochemicals and/or to provide new benefits beyond what conventional phytochemicals can provide. Examples of fungus organisms with new and useful phytochemicals relevant to the needs existing in the field are discussed below.

### SUMMARY

The present disclosure is directed to methods of crossbreeding dikaryotic fungus organisms to yield a hybrid fungus organism with a novel or enhanced phytochemical. The methods include providing a first growth medium. The methods further include placing a first dikaryotic fungus organism with a first phytochemical on the first growth medium. In an additional step, the methods include placing a second dikaryotic fungus organism with a second phytochemical on the first growth medium adjacent to the first dikaryotic fungus organism.

The presently disclosed methods further include allowing the first dikaryotic fungus organism to replicate and to form a first colony on the first growth medium. In a subsequent step, the methods include allowing the second dikaryotic fungus organism to replicate and to form a second colony on the first growth medium. The methods proceed to include allowing the first colony and the second colony to expand until they intersect along a clamp line where the first colony and the second colony exchange genetic material between them to yield a hybrid fungus organism with a novel or enhanced phytochemical.

In some examples, the method further comprises harvesting the crossbred fungus organism by removing the clamp line from the first growth medium. In such examples, the method may further comprise transferring the clamp line to a second growth medium. Moreover, such methods may further comprise allowing the clamp line to replicate and form a third colony. The method may further comprise transferring the third colony to a food source.

In some examples, the method further comprises allowing the third colony to replicate on the food source and harvesting the third colony after it has replicated on the food source. Additionally, the method may further comprise transferring the third colony to a substrate and allowing the third colony to fruit into a fruit body on the substrate.

In some examples, the substrate is manure. In other examples, the substrate is wood. In certain examples, the fruit body is a spore-producing mushroom. In select examples, the fruit body is a sporeless mushroom. In some examples, the fruit body is a truffle. In particular examples, the fruit body has an elevated concentration of one or more novel phytochemicals useful for treating cancer and Parkinson's disease.

In some examples, the first dikaryotic fungus organism is Trametes versicolor or a sub-variant thereof. The second dikaryotic fungus organism may be Ganoderma lucidum or a sub-variant thereof. The novel hybrid fungus organism may contain novel or enhanced phytochemical forms of one or more of polysaccharide and triterpenoid.

In certain examples, the first dikaryotic fungus organism is Psilocybe cyanescens or a sub-variant thereof. The second dikaryotic fungus organism may be Psilocybe cubensis or a sub-variant thereof. The novel hybrid fungus organism may contain novel or enhanced psychoactive phytochemicals. The enhanced psychoactive phytochemicals may include one or more of novel or enhanced forms of psilocybin, psilocin, and baeocystin.

### BRIEF DESCRIPTION OF THE DRAWINGS

FIG. 1 is a flow diagram of a method of crossbreeding dikaryotic fungus organisms to yield a hybrid fungus organism with a novel or enhanced phytochemical.

### DETAILED DESCRIPTION

The disclosed methods of making fungus organism hybrids with novel or enhanced phytochemicals will become better understood through review of the following detailed description in conjunction with the figures. The detailed description and FIGURES provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, a variety of examples of methods of making fungus organism hybrids with novel or enhanced phytochemicals are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given FIGURE or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

“Spore-producing mushrooms” means any normal, spore producing, physical expression type fruit body belonging to the fungi genus Psilocybe or Panaeolus.

“Sporeless mushrooms” means any normal or abnormal physical expression type fruit body belonging to the fungi genera Psilocybe or Panaeolus that lacks the ability to produce spores.

“Abnormal mutations” means any fruit body that shows any physical expression not normal to Psilocybe or Panaeolus fungi. Such mutations may include, for example, blob type mutations, fin type mutations, coral type mutations, or any fungi with mutated caps or bodies.

“Sclerotia” means the underground growing, truffle type fruit bodies that certain Psilocybe or Panaeolus species are capable of producing.

“Monokaryotic” means hyphae and mycelium that contain nuclei of one same genotype. Monokaryotic is interchangeable with heterokaryotic, homokaryotic, and uninucleate.

“Dikaryotic” means mycelium that contain binucleate cells.

“Binucleate” means cells that contain two nuclei.

“Hyphal anastomosis” means cellular fusion between branches of the same or different hyphae or mycelium.

“Hyphae” means individual cellular threads that form when spores germinate.

“Mycelium” means a collection or grouping of hyphae that have formed ropelike threads creating a web-like network.

“Culture” means a product of the cultivation of a living microorganism on a prepared nutrient medium. Such a microorganism may include, for example, Psilocybe or Panaeolus mycelium.

“Sub-variant” means a subsidiary variant or subtype of a Psilocybe or Panaeolus species. Such subtypes, for example, may include a wild Psilocybe or Panaeolus species collected from a certain location or isolated phenotypes of domesticated or wild Psilocybe or Panaeolus species.

“Domesticated species” means a Psilocybe or Panaeolus species that has been stabilized from generational selection and line breeding.

“Wild species” means a Psilocybe or Panaeolus species that has been collected from its natural growing habitat.

“Line breeding” means a form of inbreeding that involves making selections and collecting and growing spores from those selections. The inbreeding process is repeated through multiple generations so that only one or few phenotypes occur more than once within a Psilocybe or Panaeolus species sub-variant.

“Vegetatively compatible” means compatible to mate in the vegetative or non-fruiting stage of growth.

Fungus Organism Hybrids with Novel or Enhanced Phytochemicals

With reference to the FIGURES, fungus organism hybrids with novel or enhanced phytochemicals will now be described. The fungus organism hybrids will be described, in part, by methods of making them.

The fungus organism hybrids discussed herein function to provide novel or enhanced phytochemicals. Novel phytochemicals are phytochemicals that do not exist in nature. Enhanced phytochemicals are phytochemicals already known to exist, but at higher potency or in a higher concentration than they exist in nature.

The reader will appreciate from the figures and description below that the presently disclosed fungus organisms improve on conventional ways of acquiring phytochemicals and/or provide new benefits beyond what conventional phytochemicals can provide. For example, the novel fungus organisms discussed herein include novel or enhanced phytochemicals. The novel or enhanced phytochemicals provide more industrial, health, medicinal, or recreational benefits than provided by conventional phytochemicals. The novel fungus organism hybrids discussed below include novel or enhanced phytochemicals tailored to provide increased treatment potential for selected diseases and conditions.

The discussion below will first describe examples of particular fungus organism hybrids with novel or enhanced phytochemicals created by novel hyphal anastomosis fusion methods. The discussion will then describe the novel hyphal anastomosis fusion methods for creating the fungus organism hybrids with novel or enhanced phytochemicals.

### Fungus Organism Hybrid Examples

The novel hyphal anastomosis fusion methods described below have been used to create many unique crossbred fungus organism hybrids. A few selected examples of crossbred fungus organism hybrids with novel or enhanced phytochemicals produced from the methods described below are described in this section.

Trametes Versicolor-Ganoderma Lucidum Hybrid

In one specific example, an intergeneric hybrid between two fungus organisms with cancer fighting phytochemicals was created using the hyphal anastomosis fusion methods described below. The resulting intergeneric hybrid includes novel phytochemicals useful for treating cancer and Parkinson's disease.

In this example, Trametes versicolor, known commercially as Turkey Tail mushroom, was crossed with Ganoderma lucidum, known commercially as Reishi mushroom, using the methods described below to create an intergeneric hybrid Trametes versicolor-Ganoderma lucidum. Trametes versicolor contains cancer fighting polysaccharide phytochemicals and Ganoderma lucidum contains cancer fighting triterpenoid phytochemicals. The intergeneric hybrid Trametes versicolor-Ganoderma lucidum contains novel phytochemicals useful for treating cancer and Parkinson's disease.

Psilocybe cyanescens-Psilocybe cubensis Hybrid

In another specific example, an intergeneric hybrid between two fungus organisms with psychoactive phytochemicals was created using the hyphal anastomosis fusion methods described below. The resulting intergeneric hybrid includes novel or enhanced psychoactive phytochemicals.

In this example, Psilocybe cyanescens was crossed with Psilocybe cubensis using the hyphal anastomosis fusion methods described below to create an intergeneric hybrid Psilocybe cyanescens-Psilocybe cubensis. Both Psilocybe cyanescens and Psilocybe cubensis contain psychoactive phytochemicals, such as psilocybin, psilocin, baeocystin, and others. The intergeneric hybrid Psilocybe cyanescens-Psilocybe cubensis contains enhanced psychoactive psilocybin, psilocin, baeocystin, and other phytochemicals. In some instances, the intergeneric hybrid Psilocybe cyanescens-Psilocybe cubensis contains novel psychoactive phytochemicals.

Methods of Crossbreeding Fungus Organisms to Yield Fungus Organism Hybrids with Novel or Enhanced Phytochemicals

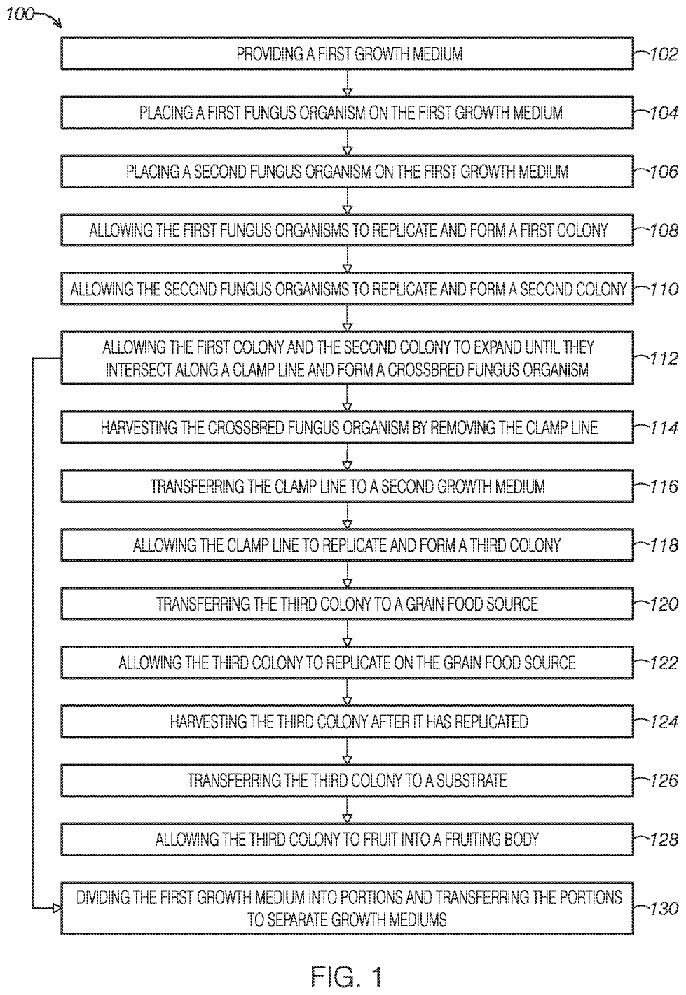

With reference to FIG. 1, methods of crossbreeding fungus organisms will now be described. The methods discussed herein function to crossbreed two distinct fungus organisms through hyphal anastomosis fusion.

With reference to FIG. 1, a first example of a method of crossbreeding fungus organisms, method 100, will now be described. Method 100 includes providing a first growth medium at step 102, placing a first fungus organism on the first growth medium at step 104, and placing a second fungus organism on the first growth medium at step 106. Step 108 of method 100 is allowing the first fungus organism to replicate to form a first colony and step 110 is allowing the second fungus organism to replicate to form a second colony.

At step 112, method 100 includes allowing the first colony and the second colony to expand until they intersect along a clamp line where the first colony and the second colony exchange genetic material between them to yield a crossbred fungus organism. Method 100 continues with harvesting the crossbred fungus organism by removing the clamp line at step 114. At step 116, method 100 includes transferring the clamp line to a second growth medium.

Method 100 further includes allowing the clamp line to replicate and form a third colony at step 118. At step 120, method 100 includes transferring the third colony to a food source. Step 122 of method 100 is allowing the third colony to replicate on the food source.

At step 124, method 100 includes harvesting the third colony after it has replicated. Step 126 of method 100 is transferring the third colony to a substrate. Method 100 further includes allowing the third colony to fruit into a fruit body at step 128.

An optional step 130 of method 100 may be taken after the first colony and the second colony intersect along a clamp line at step 112. Step 130 is dividing the first growth medium into portions and transferring the portions to separate growth mediums.

In some examples, the methods of cross breeding fungus organisms do not include one or more steps depicted in FIG. 1 for method 100. For example, some method examples do not include steps 114-130 depicted in FIG. 1. Some method examples include a subset of steps 102-130, such as steps 102-112, steps 102-114, or steps 102-116, etc. In one example, the method includes steps 102-112 and step 130. In certain examples, the method includes additional or alternative steps not included in method 100 or depicted in FIG. 1.

Providing a First Growth Medium

Providing a first growth medium at step 102 establishes a shared environment for two distinct fungus organisms to replicate and exchange genetic material through hyphal anastomosis fusion.

The first growth medium may be any currently known or later developed type of growth medium suitable for a given species of fungi. Suitable growth mediums include agar, gellen gum, liquid culture solution, grain, and any other nutrient rich media.

The size and shape of the first growth medium may be selected to suit the needs of a given application. For example, the first growth medium may be supported within a standard petri dish.

Placing Distinct Fungus Organisms on the First Growth Medium

Placing first and second fungus organisms on the first growth medium adjacent to each other at steps 104 and 106 functions to seed the first growth medium with fungus organisms to be crossbred in method 100. In the methods described herein, the first and second fungus organisms are selected from the genus Psilocybe or Panaeolus. The second fungus organism is selected to be different than the first fungus organism because method 100 functions to crossbreed two distinct fungus organisms.

In some examples, the first fungus organism is selected from the genus Psilocybe and the second fungus organism is selected from the genus Panaeolus. In other examples, the first fungus organism and the second fungus organism are both selected from the genus Psilocybe, but are different Psilocybe species or Psilocybe species variants. In still other examples, the first fungus organism and the second fungus organism are both selected from the genus Panaeolus, but are different Panaeolus species or Panaeolus species variants. In certain examples, one or more of the first fungus organism and the second fungus organism produces a sporeless fruit body.

In one particular example, the first fungus organism is Trametes versicolor, known commercially as Turkey Tail mushroom, and the second fungus organism is Ganoderma lucidum, known commercially as Reishi mushroom. In another specific example, the first fungus organism is Psilocybe cyanescens and the second fungus organism is Psilocybe cubensis.

Other fungus organism pairings tested to be suitable for the methods described herein include Psilocybe cubensis and Psilocybe semilanceata; Psilocybe cubensis and Psilocybe azurescens; Psilocybe mexicana and Psilocybe galindoi; the Golden Halo variant of Psilocybe cubensis and Psilocybe azurescens; the Golden Halo variant of Psilocybe cubensis and Psilocybe stuntzii; and the Enigma variant of Psilocybe cubensis and the Roller Coaster variant of Psilocybe cubensis. A wide range of fungus organism pairs are contemplated beyond those expressly described. Additional examples of fungus organism pairings are described below in the Specific Examples section below.

Allowing the Fungus Organisms to Replicate

Allowing the first and second fungus organisms to replicate enables the first and second colonies of the first and second fungus organisms, respectively, to form at steps 108 and 110. The first and second fungus organisms will draw nutrients from the first growth medium and naturally replicate over time.

The time, temperature, and pressure conditions for steps 108 and 110 may be selected within wide ranges as appropriate for given fungus organisms. In some examples, the fungus organisms on the growth medium are held at room temperature and atmospheric pressure until the first and second colonies have grown large enough to intersect with each other.

Allowing Colonies to Intersect

Allowing the first and second colonies to expand and intersect at step 112 serves to form a clamp line between the colonies. Forming a clamp line enables hyphal anastomosis fusion and exchanges genetic material between the first fungus organism and the second fungus organism. The exchange of genetic material yields a crossbred fungus organism.

The time required for the colonies to expand sufficiently to intersect and form a clamp line at step 112 will depend on a variety of factors. For example, different fungus organisms will have different replication rates and different growth mediums will enable different replication rates for a given fungus organism. The proximity of the two colonies on the first growth medium will also be a factor in the time needed to form a clamp line.

The time, temperature, and pressure conditions for step 112 may be selected within wide ranges as appropriate for given fungus organisms. In some examples, the fungus organisms on the growth medium are held at room temperature and atmospheric pressure until the first and second colonies have grown large enough to intersect with each other.

Dividing and Transferring Portions of the First Growth Medium

Dividing the first growth medium into portions and transferring the portions to separate growth mediums at step 130 after step 112 is an option to accelerate subsequent crossbreeding methods. Utilizing optional step 130 allows subsequent methods to effectively start new methods 100 at step 112. The new growth mediums on which portions of the first growth medium are placed will enable the already established colonies of fungus organisms from the first growth medium to continue replicating and intersecting along clamp lines. Optional step 130 may be described as anastomosis fusion.

Optional step 130 can multiply the crossbred fungus organism formed at step 112 by the divisions made of the first growth medium. For example, dividing the first growth medium into fourths and transferring the divisions to four separate growth mediums to replicate can multiply the crossbred fungus organism by approximately four times. Similarly, dividing the first growth medium into thirds and transferring the divisions to three separate growth mediums to replicate can multiply the crossbred fungus organism by approximately three times.

Harvesting and Transferring a Crossbred Fungus Organism

Harvesting the crossbred fungus organism at step 114 and transferring it to a second growth medium at step 116 serves to lay the foundation for replicating the crossbred fungus organism. Harvesting the crossbred fungus organism at step 114 is accomplished by removing the clamp line formed on the first growth medium (or on a separate growth medium receiving a division of a prior first growth medium if optional step 130 was utilized previously). Removing the clamp line may include cutting out the clamp line from a petri dish.

Transferring the clamp line to a second growth medium at step 116 establishes a new growth environment for the crossbred fungus organism to replicate. The second growth medium may be any currently known or later developed type of growth medium suitable for the crossbred fungus organism. Suitable growth mediums include agar, gellen gum, liquid culture solution, grain, and any other nutrient rich media. In some examples, the second growth medium is the same as the first growth medium.

The size and shape of the second growth medium may be selected to suit the needs of a given application. For example, the second growth medium may be supported within a standard petri dish.

Forming a Third Colony

Allowing the clamp line to replicate and form a third colony on the second growth medium at step 118 increases the quantity of the crossbred fungus organism and prepares it for further replication on a grain food source. The third colony may be allowed to grow until there is a sufficient quantity of the crossbred fungus organism and the third colony is sufficiently viable to be replicated on a grain food source.

The time required to form a sufficiently robust third colony of the crossbred fungus organism at step 118 will depend on a variety of factors. For example, different fungus organisms will have different replication rates and different growth mediums will enable different replication rates for a given fungus organism.

The time, temperature, and pressure conditions for step 118 may be selected within wide ranges as appropriate for given crossbred fungus organisms. In some examples, the crossbred fungus organism on the second growth medium is held at room temperature and atmospheric pressure until the third colony occupies a substantial majority of the second growth medium's top surface area.

Replicating the Third Colony on a Food Source

Transferring the third colony to a food source at step 120 enables the third colony to replicate extensively. The food source may be any currently known or later developed nutrient-rich food suitable for a given crossbred fungus organism. Suitable food sources for step 120 include grains, brans, and mixtures of sawdust and bran.

Allowing the third colony to replicate on the grain food source at step 122 increases the quantity of the crossbred fungus organism and prepares it for fruiting when harvested at step 124 and transferred to a substrate at step 126. The third colony may be allowed to replicate until there is a sufficient quantity of the crossbred fungus organism and the third colony is sufficiently viable to fruit when transferred to a substrate.

The time required to form a sufficiently robust third colony of the crossbred fungus organism on the food source at step 122 will depend on a variety of factors. For example, different fungus organisms will have different replication rates and different food sources will enable different replication rates for a given fungus organism.

The time, temperature, and pressure conditions for step 122 may be selected within wide ranges as appropriate for given crossbred fungus organisms. In some examples, the crossbred fungus organism on the second growth medium is held at room temperature and atmospheric pressure until the third colony occupies a substantial majority of the food source surface area.

Harvesting and Transferring the Replicated Third Colony

Harvesting the crossbred fungus organism at step 124 and transferring it to a substrate at step 126 serves to lay the foundation for fruiting the crossbred fungus organism. Harvesting the crossbred fungus organism from the food source at step 124 is accomplished by separating the food source with the crossbred fungus third colony growing on it from the excess food source that does not have significant amounts of the crossbred fungus third colony growing on it. In some examples, the entire food source is transferred to the substrate at steps 124 and 126.

Transferring the crossbred fungus organism to a substrate at step 126 establishes a fruiting environment for the crossbred fungus organism. The substrate may be any currently known or later developed type of substrate for the crossbred fungus organism. Suitable substrates include manure, wood, and soil.

The size, shape, and quantity of the substrate may be selected to suit the needs of a given application. For example, the substrate may be laid out in trays or beds.

Fruiting the Third Colony

Allowing the third colony to fruit on the substrate at step 128 creates a fruit body. The fruit body may be harvested and used for a variety of applications, including therapeutic research and treatments.

In some examples, the fruit body formed at step 128 has an elevated concentration of one or more psychoactive compounds. The psychoactive compounds with elevated concentrations may be psilocybin, psilocin, and/or baeocystin.

The form and characteristics of the fruit body will be based on the fungus organisms crossbred and may be quite varied. In some examples, the fruit body is a spore-producing mushroom. In other examples, the fruit body is a sporeless mushroom. In certain examples, the fruit body is a truffle. A wide variety of additional or alternative fruting body phenotypes and characteristics will result from crossbreeding different fungus organisms.

The time required to fruit the third colony into fruiting bodies on the substrate at step 128 will depend on a variety of factors. For example, different fungus organisms will have different fruiting rates and different substrates will enable different fruiting rates for a given fungus organism.

The time, temperature, and pressure conditions for step 128 may be selected within wide ranges as appropriate for given crossbred fungus organisms. In some examples, the crossbred fungus organism on the substrate is held at room temperature and atmospheric pressure until the fruiting bodies occupy a substantial majority of the substrate surface area.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A method of crossbreeding dikaryotic fungus organisms to yield a hybrid fungus organism with a novel or enhanced phytochemical, comprising the steps of:
providing a first growth medium;
placing a first dikaryotic fungus organism on the first growth medium, wherein the first dikaryotic fungus organism includes a first phytochemical;
placing a second dikaryotic fungus organism on the first growth medium adjacent to the first dikaryotic fungus organism, wherein the second dikaryotic fungus organism is different than the first dikaryotic fungus organism and includes a second phytochemical;
allowing the first dikaryotic fungus organism to replicate and to form a first colony on the first growth medium;
allowing the second dikaryotic fungus organism to replicate and to form a second colony on the first growth medium; and
allowing the first colony and the second colony to expand until they intersect along a clamp line where the first colony and the second colony exchange genetic material between them to yield a hybrid fungus organism with a novel or enhanced phytochemical.

2. The method of claim 1, further comprising harvesting the crossbred fungus organism by removing the clamp line from the first growth medium.

3. The method of claim 2, further comprising transferring the clamp line to a second growth medium.

4. The method of claim 3, further comprising allowing the clamp line to replicate and form a third colony.

5. The method of claim 4, further comprising transferring the third colony to a food source.

6. The method of claim 5, further comprising:
allowing the third colony to replicate on the food source; and
harvesting the third colony after it has replicated on the food source.

7. The method of claim 6, further comprising:
transferring the third colony to a substrate; and
allowing the third colony to fruit into a fruit body on the substrate.

8. The method of claim 7, wherein the substrate is manure.

9. The method of claim 7, wherein the substrate is wood.

10. The method of claim 7, wherein the fruit body is a spore-producing mushroom.

11. The method of claim 7, wherein the fruit body is a sporeless mushroom.

12. The method of claim 7, wherein the fruit body is a truffle.

13. The method of claim 7, wherein the fruit body has an elevated concentration of one or more novel phytochemicals useful for treating cancer and Parkinson's disease.

14. The method of claim 1, wherein the first dikaryotic fungus organism is Trametes versicolor or a sub-variant thereof.

15. The method of claim 14, wherein the second dikaryotic fungus organism is Ganoderma lucidum or a sub-variant thereof.

16. The method of claim 15, wherein the novel hybrid fungus organism contains novel or enhanced phytochemical forms of one or more of polysaccharide and triterpenoid.

17. The method of claim 1, wherein the first dikaryotic fungus organism is Psilocybe cyanescens or a sub-variant thereof.

18. The method of claim 17, wherein the second dikaryotic fungus organism is Psilocybe cubensis or a sub-variant thereof.

19. The method of claim 18, wherein the novel hybrid fungus organism contains novel or enhanced psychoactive phytochemicals.

20. The method of claim 19, wherein the enhanced psychoactive phytochemicals include one or more of novel or enhanced forms of psilocybin, psilocin, and baeocystin.
